(19) United States
(12) Patent Application Publication
(10) Pub. No.: US 2018/0274018 A1
(43) Pub. Date: Sep. 27, 2018

(54) RAPID PCR METHOD FOR DETECTION OF PATHOGENIC MICROORGANISMS

(71) Applicant: IMICROQ, S.L., Tarragona (ES)

(72) Inventors: Katia Uliaque Cugat, Tarragona (ES); Pablo Lozano Sánchez, Tarragona (ES); Cristina Perez Girona, Lleida (ES)

(73) Assignee: IMICROQ, S.L., Tarragona (ES)

(21) Appl. No.: 15/937,709

(22) Filed: Mar. 27, 2018

Related U.S. Application Data
(60) Provisional application No. 62/477,292, filed on Mar. 27, 2017.
(60) Provisional application No. 62/477,295, filed on Mar. 27, 2017.

(51) Int. Cl. C12Q 1/6848; C12Q 1/686

(52) U.S. Cl. CPC C12Q 1/6848; C12Q 1/686

(57) ABSTRACT

The present specification discloses methods of detecting a contaminant of interest in a sample, components useful in carrying out these methods, including PCR primers and a detection solution and kits thereof.

### CROSS REFERENCE TO RELATED APPLICATIONS

This application claims benefit of U.S. Provisional Application No. 62/477,295 and U.S. Provisional Application No. 62/477,292, each of which were filed on Mar. 27, 2017, and each of which are hereby incorporated by reference in their entirety.

### BACKGROUND

Pathogens can contaminate food or food animals during production, processing and preparation. Similarly, pathogens can contaminate water sources or seafood harvested from such contaminated waters. Common vectors include water, soil, waste or fecal matter, humans and animals. Human exposure to pathogens can cause illness, most often gastroenteritis, but also potentially more serious diseases such as salmonellosis and hepatitis A. Exposure to pathogens can occur either by direct contact with, or ingestion of, contaminated foods or water or indirectly based on cross-contamination. Even though the United States has one of the safest food supplies in the world, there are still millions of cases of foodborne illness each year.

Similarly, allergens pose a grave threat to persons with allergies or diseases related to the allergen. Such allergens include peanuts, gluten (celiac disease) lupin, tree nuts, and lactose (lactose intolerance). Another food safety issue is food adulteration, such as beef or other meats that are mixed with horse or dog meat, or illegal additives in grains, etc.

Early and rapid detection of contamination is vital to prevent widespread outbreaks of illness and ensure the health of the public at large. During the past 25 years, pathogen and allergen assays that increase sensitivity and specificity as well as decrease the time involved to perform these assays have been developed by experts in applied microbiology and microbiological analysis. Although there is no universally accepted definition for these so-called rapid assays, these methods are simply performed faster than the traditional ones, or are easier to implement, or are more sensitive and specific. Despite the name, currently available rapid assays for pathogen detection still require several days to complete the assay and determine whether a food or water supply is contaminated with a pathogen.

The main obstacles associated with reducing the time needed to conduct pathogen detection assays is the balance between the sensitivity, specificity, and lower limit of detection of the assay on one hand and having enough pathogen present to detect its presence above contaminating microorganisms and background noise.

The present specification discloses a rapid method for the detection of contaminants which provides high sensitivity and specificity, a lower limit of detection, yet can be performed more quickly than currently available methods.

### SUMMARY OF THE INVENTION

In one embodiment, the invention provides a method of detecting a selected contaminant in a sample, the method comprising the steps of:

a. combining a wash buffer with the sample and centrifuging to produce a pellet comprising the contaminant;
b. adding a lysis buffer to the pellet, incubating to lyse the contaminants cells, and centrifuging to produce nucleic acid from the contaminant in a supernatant;
c. adding a forward and a reverse primer to the supernatant and performing a PCR reaction on the nucleic acid, wherein one primer is linked to a first molecule and a second primer is linked to a second molecule;
d. incubating the PCR reaction in assay buffer with beads and an antibody that binds to the first molecule, wherein the bead is linked to a binding molecule that binds the second molecule and the antibody is linked to an enzyme, thereby forming a complex of amplified DNA, bead and antibody;
e. isolating the complex and washing the isolated complex;
f. adding to the complex a reaction buffer comprising an enzyme substrate and incubating the complex to produce a reaction mixture; and
g. analyzing an aliquot of the reaction mixture in an electrochemical sensor; wherein detection of a reaction product of the enzyme substrate in the reaction mixture indicates the presence of the selected contaminant in the sample.

In one embodiment, the contaminant is selected from the group consisting of a pathogenic microorganism, an allergan, a cross-species contamination or a food adulturation substance. In another embodiment, the pathogenic microorganism is selected from the group consisting of a bacterium, a virus, a protozoan, or a prion.

In one embodiment, the virus is a member of a family selected from the group consisting of Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, Togaviridae, Anelloviridae, Caliciviridae, Filoviridae, Bornaviridae, Reoviridae, Bunyaviridae, Arenaviridae, Coronaviridae, Astroviridae, Arteriviridae, Hepeviridae.

In one embodiment, the virus is Hepatitis A, norovirus or rotavirus.

In one embodiment, the bacteria belongs to the genus Bacillus, Bordetella, Borrelia, Brucella, Campylobacter, Chlamydia, Chlamydophila, Clostridium, Corynebacterium, Enterobacter, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia.

In one embodiment, the bacterium is selected from the group consisting of Listeria monocytogenes, Salmonella typhimurium, Salmonella enteritidis, Campylobacter jejuni, Cronobacter sp., and Escherichia coli.

In one embodiment, the protozoan belongs to the genus Acanthamoeba, Balamuthia, Cryptosporidium, Dientamoeba, Endolimax, Entamoeba, Giardia, Iodamoeba, Leishmania, Naegleria, Plasmodium, Sappinia, Toxoplasma, Trichomonas, or Trypanosoma.

In one embodiment, the protozoan is Toxoplasmosis gondii, Giardia lamblia, Entamoeba histolytica, or Cryptosporidium parvum.

In one embodiment, the allergen is selected from the group consisting of peanuts, gluten, lupin, tree nuts, and lactose.

In one embodiment, the bead is magnetic and the complex is isolated with a magnet.

In one embodiment, the lysis buffer comprises 5 mM NaOH and 0.25% SDS.

In another embodiment, the lysis buffer is composed of 2.5 mL SDS 10%, 5 mL NaOH 1N, and 92.5 mL H2O MilliQ.

In one embodiment, the first molecule is digoxigenin and the antibody is an anti-digoxigenin antibody.

In one embodiment, the second molecule is biotin and the second binder is streptavidin.

In one embodiment, the enzyme is alkaline phosphatase and the substrate is para-amino phenyl phosphate.

In one embodiment, the sample is a food sample or a livestock sample. In one embodiment, the livestock sample is chicken broiler feces or a chicken layer feces. In one embodiment, the food sample is a chicken egg.

In one aspect, the invention provides a method of detecting a contaminant in a sample, the method comprising the steps of:

a. combining a wash buffer with the sample and centrifuging to produce a pellet comprising the contaminant;
b. adding a lysis buffer to the pellet, incubating to lyse the cells, and centrifuging to produce nucleic acid from the contaminant in a supernatant;
c. adding a forward and a reverse primer to the supernatant and performing a PCR reaction on the nucleic acid, wherein one primer is linked to biotin and one primer is linked to digoxigenin;
d. incubating the PCR reaction in assay buffer with beads and an anti-digoxigenin antibody, wherein the bead is linked to streptavidin and the antibody is linked to alkaline phosphatase, thereby forming a complex of amplified DNA, bead and antibody;
e. isolating the complex and washing the isolated complex;
f. adding to the complex a reaction buffer comprising para-amino phenyl phosphate and incubating the complex to produce a reaction mixture; and
g. analyzing an aliquot of the reaction mixture in an electrochemical sensor; wherein detection of para-amino phenyl in the reaction mixture indicates the presence of the selected contaminant in the sample.

### BRIEF DESCRIPTION OF THE DRAWINGS

FIG. 1 shows the complex of the assay for detection of the pathogen 100, where the complex is formed from the labeled PCR primers, which are then sandwiched by streptavidin labeled beads and anti-digoxigenin antibodies linked to alkaline phosphatase.

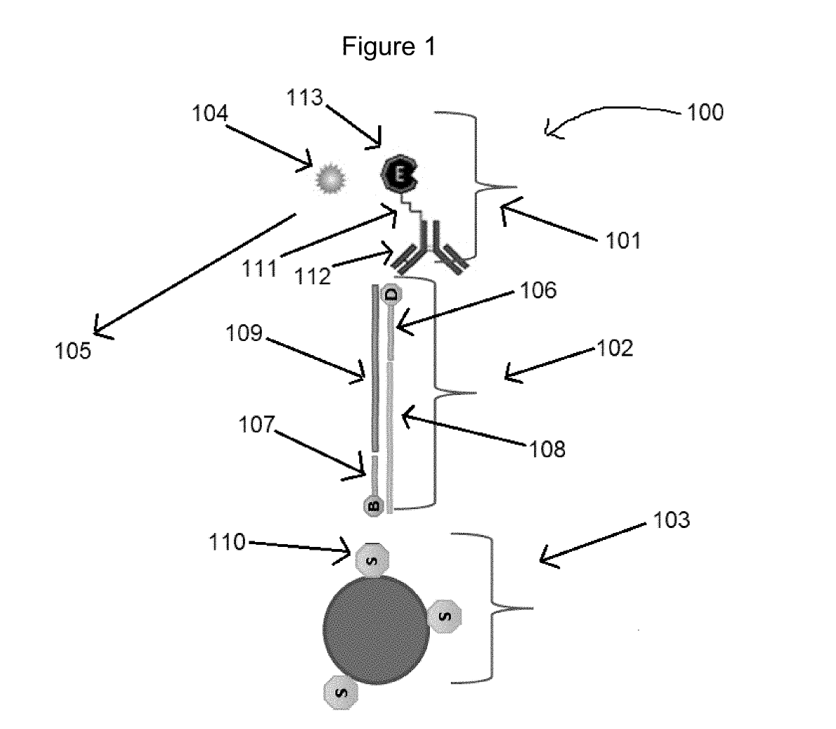

### DETAILED DESCRIPTION OF THE INVENTION

The present specification discloses a method of detecting a contaminant in a sample. The method comprises PCR amplification of DNA from a sample, such as a food or animal sample, using contaminant-specific primers. The primers 106 and 107 are linked to one member of a binding pair 102 such as biotin 109 and a second molecule such as digoxigenin 108, which are then captured in a sandwich complex 101 and 103 using an antibody 112 (e.g., anti-digoxigenen) linked 111 to an enzyme 113 (e.g., alkaline phosphatase) and a bead 103 linked to the second member of the binding pair 107, e.g., streptavidin 110. The beads (and the complex) are isolated mechanically (preferably using magnetic beads and a magnet). The complex is incubated with an enzyme substrate 104, e.g., para-amino phenyl phosphate, which in the presence of the enzyme, e.g., alkaline phosphatase converts to the reaction products, e.g., para-amino phenyl and phosphate. An electrochemical sensor 105 is used to detect the reaction product, e.g., phosphate and thus the presence of the contaminant in the sample. The use of PCR allows for a more sensitive and precise detection of the contaminant then currently available methods. In addition, the method disclosed herein allows for a more rapid detection of a contaminant since it may be completed in a matter of hours; current pathogen/contaminant detection methods require about 2 days to about 5 days to complete. Finally, the use of PCR and an electrochemical sensor provides for a much more cost-effective method of detection, in comparison with currently available methods.

A contaminant in a sample, such as a food or livestock sample, is a undesirable, unwanted substance that is not suitable for human consumption as it can cause disease or an allergic reaction, or is a foreign substance that is not a labeled component of the food, e.g., an adulteration of the food by a cheaper or substitute substance such as a different species of meat, grain, or a colorant or flavor.

The definition of a pathogenic organism or microorganism is an organism capable of causing disease in its host. A human pathogen is capable of causing illness in humans. Non-limiting examples of a pathogen include a prion, a virus, a bacterium, a fungus, a protazoan, a helimenth, and a parasite. Common examples of pathogenic microorganisms include specific strains of bacteria like Salmonella (e.g., Salmonella enteritidis, Salmonella typhimurium), Listeria (e.g., Listeria monocytogenes) and Escherichia coli (e.g., enterotoxigenic, enteropathogenic, and enterohemorrhagic strains); viruses such as Hepatitis A, norovirus, and rotavirus; protozoa such as Cryptosporidium parvum, Toxoplasmosis gondii, and Giardia lamblia; and prions.

A prion is composed of a protein in a misfolded form. Prions reproduce by hijacking the functions of living cells and propagating a misfolded protein state by inducing existing, properly-folded proteins to convert into the disease-associated, prion form. As such, the prion acts as a template to guide the misfolding of more proteins into prion form. These newly formed prions can then go on to convert more proteins themselves, which triggers a chain reaction that produces large amounts of the prion form. Prions cause neurodegenerative disease by aggregating extracellularly within the central nervous system to form plaques known as amyloid, which disrupt the normal tissue structure. Prions are responsible for the transmissible spongiform encephalopathies in a variety of mammals, including bovine spongiform encephalopathy (BSE, also known as “mad cow disease”) in cattle, scrapies in sheep and goats, and chronic wasting disease in deer. In humans, prions cause Creutzfeldt-Jakob disorders, Gerstmann-Sträussler-Scheinker syndrome, Fatal Familial Insomnia, and kuru.

A virus is a small infectious agent typically range between 20-300 nanometers in length that replicates only inside the living cells of other organisms. Virus particles (known as virions) consist of two or three parts: i) the genetic material made from either DNA or RNA, long molecules that carry genetic information; ii) a protein coat that protects these genes; and in some cases iii) an envelope of lipids that surrounds the protein coat when they are outside a cell. The shapes of viruses range from simple helical and icosahedral forms to more complex structures. The average virus is about one one-hundredth the size of the average bacterium. Viruses can infect all types of life forms, from animals and plants to bacteria and archaea. Non-limiting examples of pathogenic viruses belong to the families Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae.

A bacteria is a single-celled prokaryotic microorganism characterized by the lack of a membrane-bound nucleus and membrane-bound organelles and can have a cell wall (Gram positive) or lack one (Gram negative). Morphologically, bacteria can be divided into rod-shaped (bacillus), round (coccus), spiral (spirillum), and incomplete spiral (vibrios). Non-limiting examples of pathogenic bacteria belong to the genera Bacillus, Bordetella, Borrelia, Brucella, Campylobacter, Chlamydia, Chlamydophila, Clostridium, Corynebacterium, Enterobacter, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia. Non-limiting examples of specific pathogenic bacterial species include a strain of Bacillus anthracis, a strain of a strain of Bordetella pertussis, a strain of a strain of Borrelia burgdorferi, a strain of a strain of Brucella abortus, a strain of a strain of Brucella canis, a strain of a strain of Brucella melitensis, a strain of a strain of Brucella suis, a strain of a strain of Campylobacter jejuni, a strain of Chlamydia pneumonia, a strain of Chlamydia trachomatis, a strain of Chlamydophila psittaci, a strain of Clostridium botulinum, a strain of Clostridium difficile, a strain of Clostridium perfringens, a strain of Clostridium tetani, a strain of Corynebacterium diphtheria, a strain of Enterobacter sakazakii, a strain of Enterococcus faecalis, a strain of Enterococcus faecium, a strain of Escherichia coli, a strain of Francisella tularensis, a strain of Haemophilus influenza, a strain of Helicobacter pylori, a strain of Legionella pneumophila, a strain of Leptospira interrogans, a strain of Listeria monocytogenes, a strain of Mycobacterium leprae, a strain of Mycobacterium tuberculosis, a strain of Mycobacterium ulcerans, a strain of Mycoplasma pneumonia, a strain of Neisseria gonorrhoeae, a strain of Neisseria meningitides, a strain of Pseudomonas aeruginosa, a strain of Rickettsia rickettsia, a strain of Salmonella typhi and Salmonella typhimurium, a strain of Shigella sonnei, a strain of Staphylococcus aureus, a strain of Staphylococcus epidermidis, a strain of Staphylococcus saprophyticus, a strain of Streptococcus agalactiae, a strain of Streptococcus pneumonia, a strain of Streptococcus pyogenes, a strain of Treponema pallidum, a strain of Vibrio cholera, a strain of Yersinia enterocolitica, and a strain of Yersinia pestis.

A bacterial strain can be a pathogenic strain or a non-pathogenic strain. A pathogenic bacterial strain is one that causes or facilitates a disease, infection or other adverse effect in a mammal. A pathogenic bacterial strain is considered to be an abnormal or infectious flora. A non-pathogenic bacterial strain is one that is considered harmless to a mammal because no appreciable disease, infection or other adverse effect is associated with the presence of the non-pathogenic bacterial strain in the mammal. A non-pathogenic bacterial strain is considered to be a benign or beneficial flora. A non-pathogenic bacterial strain includes a live vaccine bacterial strain. A live vaccine bacterial strain is typically an attenuated bacterial strain used to elicit an immune response that creates protective antibodies in an individual, such as poultry, livestock or humans, against pathogenic bacterial strains (or bacterial field strains) that cause infections. In an embodiment, live vaccine bacterial strain includes a Salmonella live vaccine strain. Examples of a Salmonella live vaccine bacterial strain include, without limitation, a Salmonella typhimorium live vaccine strain and Salmonella enteritidis live vaccine strain.

A fungus is a eukaryotic microorganism characterized by membrane-bound nucleus and organelles which lack chlorophyll have cell walls composed of chitin and reproduce by spores. Non-limiting examples of pathogenic fungi belong to the genera Aspergillus, Candida, Cryptococcus, Histoplasma, Pneumocystis, and Stachybotrys. Non-limiting examples of specific pathogenic fungi species include a strain of Aspergillus clavatus, Aspergillus fumigatus, Aspergillus flavus, Candida albicans, Cryptococcus albidus, Cryptococcus gattii, Cryptococcus laurentii, Cryptococcus neoformans, Histoplasma capsulatum, Pneumocystis jirovecii, Pneumocystis carinii, and Stachybotrys chartarum.

A protozoa is a eukaryotic single-cell microorganism characterized by membrane-bound nucleus and organelles which lack chlorophyll and a cell wall and are motile. Protozoa commonly range from 10 to 52 micrometers, but can grow as large as 1 mm. Non-limiting examples of pathogenic protozoa belong to the genera Acanthamoeba, Balamuthia, Cryptosporidium, Dientamoeba, Endolimax, Entamoeba, Giardia, lodamoeba, Leishmania, Naegleria, Plasmodium, Sappinia, Toxoplasma, Trichomonas, and Trypanosoma. Non-limiting examples of specific pathogenic protozoa species include a strain of Acanthamoeba spp., Balamuthia mandrillaris, Cryptosporidium canis, Cryptosporidium fells, Cryptosporidium hominis, Cryptosporidium meleagridis, Cryptosporidium muris, Cryptosporidium parvum, Dientamoeba fragilis, Endolimax nana, Entamoeba dispar, Entamoeba hartmanni, Entamoeba histolytica, Entamoeba coli, Entamoeba moshkovskii, Giardia lamblia, lodamoeba butschlii, Leishmania aethiopica, Leishmania braziliensis, Leishmania chagasi, Leishmania donovani, Leishmania infantum, Leishmania major, Leishmania mexicana, Leishmania tropica, Naegleria fowleri, Plasmodium falciparum, Plasmodium knowlesi, Plasmodium malariae, Plasmodium ovale, Plasmodium vivax, Sappinia diploidea, Toxoplasma gondii, Trichomonas vaginalis, Trypanosoma brucei, and Trypanosoma cruzi.

Aspect of the present specification disclose, in part, a sample. A variety of samples are useful in the methods disclosed herein. A sample refers to a biological matter that contains or potentially contains a pathogen. A sample encompasses but is not limited to a food sample, livestock such as chicken (broiler or layer) feces, a product of the livestock, such as an egg, purified pathogen, a partially purified pathogen, a cell, a crude cell lysate, a partially purified cell lysate, a crude culture media, a partially purified culture media, a raw foodstuff (e.g., a foodstuff such as a vegetable, fruit, grain, nut, spice, legume, dairy, egg, meat, fish, shellfish, or a beverage made from such items), a partially-cooked foodstuff, a cooked foodstuff, a processed foodstuff; a dairy foodstuff, a beverage, an animal feed, a fecal sample, a vegetative sample, a soil sample, a water sample, a pond sediment, a human tissue sample, a raw livestock tissue sample, a processed livestock tissue sample, such as, e.g., leather. The present invention in one embodiment provides a rapid test for food samples, either for preventive purposes or for diagnostic purposes.

In the present invention, detection presence or absence of a pathogen of interest occurs using a nucleic acid-based detection method. Non-limiting examples of nucleic acid-based detection method include DNA-based detection methods and RNA-based detection methods such as PCR-based assays and RT-PCR-based assays. The invention detects the presence or absence amplified nucleic acid using a electrochemical sensor-based detection method.

In the invention, the PCR primers are each linked to a different molecule. One PCR primer is linked to a first member of a binding pair. In one embodiment, the first member of the binding pair is biotin and its counterpart is streptavidin. Other binding pairs are disclosed in Bayer et al., J. Chromatography, 510:3-11 (1990), herein incorporated by reference in its entirety. The second PCR primer is linked to a molecule that has an antibody as its binding partner. Any suitable antigen can serve as the molecule linked to the primer.

Either the antibody or the non-PCR linked member of the binding pair is linked to an enzyme that reacts with a substrate to form a detectable reaction product, the reaction product typically detected using an electrochemical sensor, although other forms of detection are envisioned, such as fluorescent, colorimetric, etc. Exemplary enzymes include kinases, phosphatases, lipases, phospholipases, and saccharide cleaving enzymes, e.g., alkaline phosphatase, luciferase, horseradish peroxidase, lactase, and β-galactosidase, and their substrates and modified substrates.

In one embodiment, an aliquot of a sample as described above is transferred into a container and is centrifuged. In one embodiment, 1 mL of the sample is transferred. If the sample is solid, it is dissolved or soaked in water or buffer first. After centrifugation, the supernatant is discarded and a lysis buffer is added to the pellet. In one embodiment, the lysis buffer is 0.25% SDS and 5 mM NaOH. In one embodiment, the amount of lysis buffer is 250 μL. The sample in lysis buffer is incubated at a suitable temperature for a suitable length of time, in one embodiment, at 98° C. for 30 minutes.

The lysis buffer tube is centrifuged and an aliquot is transferred to a dilution solution. In one embodiment, a 10 μL aliquot is transferred to 990 μL of dilution solution (typically water or an isotonic buffer). Then an aliquot of the dilution solution is transferred to an amplification solution comprising selected primers for the contaminant of choice, in one embodiment about 10 μM each of the forward and reverse primers (one linked to biotin and one to digoxigenin). The amplification or PCR solution also contains nucleotides, enzyme, buffer and other components of a PCR reaction. In one embodiment, about 5 μL to about 15 μL of the dilution solution is transferred to the PCR reaction. The reaction is incubated for about 48 minutes at cycling temperatures suitable for the primers in the mixture to amplify the target nucleotide sequence from the contaminant.

After amplification, an aliquot of the PCR product is combined with capture solution (streptavidin beads in stock solution), reporter solution (anti-digoxigenin antibody linked to alkaline phosphate in solution), and assay buffer. In one embodiment, the reporter solution comprises 0.05M TRIS pH 8.0, 1% BSA, 50% glycerol, 0.001 M MgCl₂, 0.015 M sodium azide and 1.5 mg/ml beads. In one embodiment, the assay buffer comprises 2% BSA, 10% TWEEN 20, 0.005 M EDTA, 0.1 M TRIS, and 0.15 M NaCl, pH 7.5. In one embodiment, 20 μL of PCR solution is combined with 3 μL of capture solution, 50 μL reporter solution and 120 μL assay buffer. The reaction is incubated at a suitable temperature and time with a magnet. In one embodiment, the temperature is 37° C. for 10 minutes. After incubation, the mixture is exposed to a magnet and the supernatant is removed, leaving the magnetic beads.

The magnetic beads are washed with a wash buffer. The wash buffer can be any suitable isotonic buffer, as described below. In one embodiment, the wash buffer is 0.01 M PBS, 0.138 M NaCl, 0.00027 M KCL at pH 7.4. In one embodiment, 1 mL of wash buffer is used. This step may be repeated.

The supernatant (wash buffer) is removed, and a reaction solution is added to the beads. In one embodiment, 20 μL of reaction solution is added to the beads. The reaction buffer typically contains p-amino phenyl phosphate and a buffer. In one embodiment, the buffer is TRIS, e.g., 0.2 M TRIS pH 8.2 with 1 mM p-amino phenyl phosphate. The reaction solution is incubated at a suitable time and temperature. In one embodiment, the reaction is incubated at 37° C. for 10 minutes.

The reaction is analyzed in a electrochemical sensor, which detects cleavage of phosphate from p-amino phenyl phosphate by the alkaline phosphatase enzyme that is complexed with the contaminant DNA. An aliquot of the reaction solution, in one embodiment 10 μL, is analyzed using the electrochemical sensor. Detection of para-aminophenol above a control or baseline sample indicates presence of the contaminant.

Any buffer may be used above, with the proviso that the resulting buffered solution is useful to practice the methods disclosed herein. A buffered solution can be varied as appropriate by one skilled in the art. Therefore, aspects of this embodiment may optionally include, e.g., 2-(N-morpholino) ethanesulfonic acid (MES), N-(2-acetamido)iminodiacetic acid (ADA), dimethylarsinic acid (Cacodylate), piperazine-N, N′-bis(2-ethanesulfonic acid) (PIPES), N-(2-Acetamido)-2-aminoethanesulfonic acid (ACES), cholamine chloride, N,N′-bis(2-hydroxyethyl)-2-aminoethanesulfonic acid (BES), 3-(N-morpholino) propanesulfonic acid (MOPS), 2-{[tris(hydroxymethyl)methyl]amino} ethanesulfonic acid (TES), N-(2-hydroxyethyl) piperazine-N′-(2-ethanesulfonic acid) (HEPES), piperazine-N,N′-bis(2-hydroxypropanesulfonic acid) (POPSO), N-tris(hydroxymethyl) methylglycine (Tricine), tris(hydroxymethyl)methylamine (Tris), acetamidoglycine, N,N-bis(2-hydroxyethyl)glycine (Bicine), N-tris(hydroxymethyl)methyl-3-am inopropanesulfonic acid (TAPS), 3-[(1,1-dimethyl-2-hydroxyethyl)amino]-2-hydroxypropanesulfonic acid (AMPSO), 3-(cyclohexylamino)-2-hydroxy-1-propanesulfonic acid (CAPSO), and 3-(cyclohexylamino)-1-propanesulfonic acid (CAPS); acetate buffers, such as, e.g., magnesium acetate, potassium acetate, and Tris acetate; borate buffers; citrate buffers; phosphate buffers, such as, e.g., potassium phosphate buffers and sodium phosphate buffers; saline buffers, such as, e.g., phosphate-buffered saline (PBS), HEPES-buffered saline (HBS), and Tris-buffered saline (TBS), saline sodium citrate (SSC); universal buffers, such as, e.g., buffers comprising citric acid and potassium phosphate, Britton-Robinson buffer, Carmody buffer and the like, or any combination thereof. Non-limiting examples of how to make and use specific buffers are described in, e.g., MOLECULAR CLONING, A LABORATORY MANUAL (Joseph Sambrook & David W. Russell eds., Cold Spring Harbor Laboratory Press, 3rd ed. 2001) and CURRENT PROTOCOLS IN MOLECULAR BIOLOGY (Frederick M. Ausubel et al., eds. John Wiley & Sons, 2004).

Any temperature may be used during incubation of a lysis, amplification, assay, or reaction solution, with the proviso that the temperature is useful to practice the methods disclosed herein. In aspects of this embodiment, a temperature used may be, e.g., about 15° C., about 16° C., about 17° C., about 18° C., about 19° C., about 20° C., about 21° C., about 22° C., about 23° C., about 24° C., or about 25° C., 37° C., 45° C., 50° C., 55° C., 60° C., 65° C., 70° C., 75° C., 80° C., 85° C., 90, 91° C., 92° C., 93° C., 94° C., 95° C., 96° C., 97° C., 98° C., 99° C., or 100° C.

Any time may be used during incubation of a solution, with the proviso that the time is useful to practice the methods disclosed herein. In aspects of this embodiment, a time used to incubate may be, e.g., about 5 minutes, about 10 minutes, about 15 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, or about 150 minutes.

An electrochemical signal can be analyzed using an instrument which is capable of measuring and/or analyzing potentiometric, voltammetric, amperometric and/or impedance/conductance parameters. Typically, such instrumentation is operated using computer-controlled software. A non-limiting example is PalmSens3, a potentiostat, galvanostat, and impedance analyzer and PSTrace, its accompanying software (PalmSens BV, Utrecht, Netherlands). In another embodiment, computer and tablet systems are used with integrated firmware and software.

Aspects of the present specification disclose, in part, a contaminant analysis kit to carry out the method of detecting a contaminant disclosed herein. A contaminant analysis kit disclosed herein contains components necessary for the detection of the pathogen of interest. In some aspects, a contaminant analysis kit disclosed herein components necessary for the detection of a single contaminant of interest. In some aspects, a contaminant analysis kit disclosed herein components necessary for the detection of a plurality of contaminant of interest.

In some embodiment, a contaminant analysis kit disclosed herein typically comprises one or more of a wash buffer, a lysis buffer, PCR primers (one primer linked to biotin and one primer linked to digoxigenin) amplification solution, assay buffer, capture solution (containing streptavidin linked beads, preferably magnetic beads), reporter solution (containing an anti-digoxigenin antibody linked to alkaline phosphatase) and reaction solution comprising p-amino phenyl phosphate.

A contaminant analysis kit disclosed herein may further comprise an instrument which is capable of measuring and/or analyzing potentiometric, voltam metric, amperometric and/or impedance/conductance parameters.

A contaminant analysis kit disclosed herein may further comprise a suitable container, for example, a vessel, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the agent (e.g., a vessel), composition and any other reagent containers in close confinement for commercial sale. Such containers may include injection or blow-molded plastic containers into which the desired vials are retained.

A contaminant analysis kit disclosed herein may further comprise a labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk, flash memory), optical disk such as CD- or DVD-ROM/RAM, DVD, MP3, magnetic tape, or an electrical storage media such as RAM and ROM or hybrids of these such as magnetic/optical storage media, FLASH media or memory type cards. Labels or inserts may include identifying information of one or more components therein, amounts used for one or more components therein, step by step instructions of how to perform a method of detecting a pathogen of interest. Labels or inserts can include information identifying manufacturer information, lot numbers, manufacturer location and date and patent information.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A method of detecting a selected contaminant in a sample, the method comprising the steps of:
a. combining a wash buffer with the sample and centrifuging to produce a pellet comprising the contaminant;
b. adding a lysis buffer to the pellet, incubating to lyse the contaminants cells, and centrifuging to produce nucleic acid from the contaminant in a supernatant;
c. adding a forward and a reverse primer to the supernatant and performing a PCR reaction on the nucleic acid, wherein one primer is linked to a first molecule and a second primer is linked to a second molecule;
d. incubating the PCR reaction in assay buffer with beads and an antibody that binds to the first molecule, wherein the bead is linked to a binding molecule that binds the second molecule and the antibody is linked to an enzyme, thereby forming a complex of amplified DNA, bead and antibody;
e. isolating the complex and washing the isolated complex;
f. adding to the complex a reaction buffer comprising an enzyme substrate and incubating the complex to produce a reaction mixture; and
g. analyzing an aliquot of the reaction mixture in an electrochemical sensor;
wherein detection of a reaction product of the enzyme substrate in the reaction mixture indicates the presence of the selected contaminant in the sample.

2. The method of claim 1, wherein the contaminant is selected from the group consisting of a pathogenic microorganism, an allergan, a cross-species contamination or a food adulturation substance.

3. The method of claim 2, wherein the pathogenic microorganism is selected from the group consisting of a bacterium, a virus, a protozoan, or a prion.

4. The method of claim 3, wherein the virus is a member of a family selected from the group consisting of Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, Togaviridae, Anelloviridae, Caliciviridae, Filoviridae, Bornaviridae, Reoviridae, Bunyaviridae, Arenaviridae, Coronaviridae, Astroviridae, Arteriviridae, Hepeviridae.

5. The method of claim 4, wherein the virus is Hepatitis A, norovirus or rotavirus.

6. The method of claim 3, wherein the bacteria belongs to the genus Bacillus, Bordetella, Borrelia, Brucella, Campylobacter, Chlamydia, Chlamydophila, Clostridium, Corynebacterium, Enterobacter, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia.

7. The method of claim 6, wherein the bacterium is selected from the group consisting of Listeria monocytogenes, Salmonella typhimurium, Salmonella enteritidis, Campylobacter jejuni, Cronobacter sp., and Escherichia coli.

8. The method of claim 3, wherein the protozoan belongs to the genus Acanthamoeba, Balamuthia, Cryptosporidium, Dientamoeba, Endolimax, Entamoeba, Giardia, Iodamoeba, Leishmania, Naegleria, Plasmodium, Sappinia, Toxoplasma, Trichomonas, or Trypanosoma.

9. The method of claim 8, wherein the protozoan is Toxoplasmosis gondii, Giardia lamblia, Entamoeba histolytica, or Cryptosporidium parvum.

10. The method of claim 2, wherein the allergen is selected from the group consisting of peanuts, gluten, lupin, tree nuts, and lactose.

11. The method of claim 1, wherein the bead is magnetic and the complex is isolated with a magnet.

12. The method of claim 1, wherein the lysis buffer comprises 5 mM NaOH and 0.25% SDS.

13. The method of claim 1, wherein the first molecule is digoxigenin and the antibody is an anti-digoxigenin antibody.

14. The method of claim 1, wherein the second molecule is biotin and the second binder is streptavidin.

15. The method of claim 1, wherein the enzyme is alkaline phosphatase and the substrate is para-amino phenyl phosphate.

16. The method of claim 1, wherein the sample is a food sample or a livestock sample.

17. The method of claim 16, wherein the livestock sample is chicken broiler feces or a chicken layer feces.

18. The method of claim 16, wherein the food sample is a chicken egg.

19. A method of detecting a contaminant in a sample, the method comprising the steps of:
a. combining a wash buffer with the sample and centrifuging to produce a pellet comprising the contaminant;
b. adding a lysis buffer to the pellet, incubating to lyse the cells, and centrifuging to produce nucleic acid from the contaminant in a supernatant;
c. adding a forward and a reverse primer to the supernatant and performing a PCR reaction on the nucleic acid, wherein one primer is linked to biotin and one primer is linked to digoxigenin;
d. incubating the PCR reaction in assay buffer with beads and an anti-digoxigenin antibody, wherein the bead is linked to streptavidin and the antibody is linked to alkaline phosphatase, thereby forming a complex of amplified DNA, bead and antibody;
e. isolating the complex and washing the isolated complex;
f. adding to the complex a reaction buffer comprising para-amino phenyl phosphate and incubating the complex to produce a reaction mixture; and
g. analyzing an aliquot of the reaction mixture in an electrochemical sensor;
wherein detection of para-amino phenyl in the reaction mixture indicates the presence of the selected contaminant in the sample.
